(19) United States
(12) Patent Application Publication
(10) Pub. No.: US 2017/0042742 A1
(43) Pub. Date: Feb. 16, 2017

(54) ARTICLE OF MANUFACTURE PRESSURE RELIEVING PAD

(71) Applicant: Innovative Medical Products, Inc., Plainville, CT (US)

(72) Inventor: Edward DEMAYO, Greenbrae, CA (US)

(73) Assignee: Innovative Medical Products, Inc., Plainville, CT (US)

(21) Appl. No.: 15/233,909

(22) Filed: Aug. 10, 2016

Related U.S. Application Data
(60) Provisional application No. 62/203,885, filed on Aug. 11, 2015.

(51) Int. Cl. A61F 13/04; A61F 5/32; A61F 5/058

(52) U.S. Cl. CPC A61F 13/046; A61F 5/058; A61F 5/32; A61F 2013/00489

(57) ABSTRACT

An article of manufacture for a bony prominence under casts, splints and or other devices utilized to hold the limb in a fixed position for an extended period of time. The article consists of a pressure relieving pad from a base substrate with a predetermined design formed from a gel material with an adhesive quality. In another embodiment, the article consists of a pressure relieving pad formed from a gel material base substrate combined with a fibrous substrate with a predetermined design of the pressure relieving pad. Each article includes at least one ventilation channel cut through the base substrate and/or fibrous substrate, e.g. the ventilation channel may have a single or multiple cut incision. The ventilation channel functioning to increase the padding around the base of the prominence thereby providing extended comfort and fitting of a rigid splint, cast or other fixing device.

### CROSS-REFERENCE TO RELATED APPLICATION

This application claims the benefit of U.S. Provisional Application No. 62/203,885, filed Aug. 11, 2015 entitled “Article of Manufacture Pressure Relieving Star Pad,” which is incorporated in its entirety.

### FIELD OF THE INVENTION

The present invention relates to an article of manufacture for treating fractures and soft tissue injuries and, more importantly, to an article of manufacture in the form of a pressure relieving pad for treating bony prominences when a cast, splint or other device for stabilizing the underlying injury.

### BACKGROUND OF THE INVENTION

Conventional systems and methods for treating fractures and soft tissue injuries of a limb or other extremity of the human body include casts, splints or other devices for stabilizing the underlying injury. Typically, an external support is used that can take various forms and designs such as, for example, casts and splints made of plaster or fiberglass materials. In operation, the underlying injury is stabilized by a cast or splint closely fitting to the limb or other body part in order to avoid excessive motion. The cast or splint must remain rigid over an extended period of time while the underlying fracture and/or soft tissue injury heals. Problems occur during this extended period of time of feeling as the rigid cast or splint may irritate, cause pain, and/or otherwise be uncomfortable to the patient.

In order to make extended period of time more comfortable for the patient, conventional treatments have recently included a thin wrap of poly type material between the skin and the cast. However, while this may bring some comfort, irritation and pain and discomfort still occur as bony prominences of the foot will rub against the cast, e.g. as the body part and/or limb atrophies forms between the bony prominence and the cast and with movement causes discomfort.

Conventional techniques have applied fabric padding of synthetic and natural materials of uniform thickness under the cast so as to provide comfort and avoid excessive pressure on underlying bony prominences. Conventional techniques add wrapping around the bony prominences by using additional wraps or layers of the padding material. However, problems and discomfort to the patient can still occur because such fabric padding may result in excessive looseness in the cast—both initially and over time as the limb atrophies.

Moreover, use of additional wraps and/or layers of padded material in castings and splints have a tendency to compact over the bony prominence, resulting in increased pressure on the bony prominence causing increased discomfort and, possibly, a need to replace the cast thereby increasing the healthcare cost, cost of materials, and time of treatment with the physician and/or nurse practitioner. A problem has been observed that using extra wrapping and/or padding material over the bony prominence builds up the maximum thickness directly over the prominence, thereby putting pressure and/or creating a pressure point directly on the bony prominence.

Consequently, and improved, better approach is needed to address a long felt need in treating fractures and or soft tissue injuries using splints, casts and/or other devices to hold the underlying limb in a fixed position for an extended period of time that improves the patient comfort over such extended period of time.

### SUMMARY OF THE INVENTION

Accordingly it is an object of the present invention to provide a new and improved approach to providing patient comfort in the area of a bony prominence under casts, splints and or other devices utilized to hold the limb in a fixed position for an extended period of time.

The present invention is directed to an article of manufacture, system and method of increasing the padding around the base of the prominence, thereby spreading the pressure over a greater surface area.

The present invention is an article of manufacture utilizing gel pressure relieving pad with an adhesive quality as the pressure pad alone or combined with a fibrous substrate with a predetermined design of the pressure relieving pad and cut through the gel and fibrous substrate (e.g. a single or multiple cut or incision) functioning to increase the padding around the base of the prominence thereby providing extended comfort and fitting of a rigid splint, cast or other fixing device.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 illustrates a top view of the pressure relieving pad in accordance with an embodiment of the present invention; and

FIG. 2 illustrates a side view of the pressure relieving pad in accordance with an embodiment of the present invention;

FIG. 3 illustrates a perspective view of the pressure relieving pad adjacent a bony prominence of the foot; and

FIG. 4 illustrates a perspective view of the pressure relieving pad on a bony prominence of the foot in accordance with an embodiment of the present invention.

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically. The term “approximately” or “substantially” is defined as reasonably close and a range of proportions which is disclosed and claimed as what was regarded as the invention is not rendered indefinite by the qualification “approximately” or “substantially.”

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent, e.g. differs from the claimed limitation only insubstantially, in view of the disclosure herein and use of the term “means” is not intended to be limiting.

The term “cast” refers to a shell, frequently made from plaster, encasing a limb (or, in some cases, large portions of the body) to stabilize and hold anatomical structures, most often a broken bone (or bones), in place until healing is confirmed. The cast functions similar to a splint. Casts may also be referred to in specific situations as an orthopedic cast, body cast, plaster cast, or surgical cast. Typical constructions of casts include plaster bandages consist essentially of (1) a cotton bandage that has been combined with plaster of paris, which hardens after it has been made wet, (2) synthetic materials consisting essentially of knitted fiberglass bandages impregnated with polyurethane, or (3) bandages of thermoplastic. Synthetic bandages have advantages as being lighter and dry much faster than plaster bandages. Plaster bandages have advantages as plaster can be more easily molded to make a snug and therefore more comfortable fit as well as a plaster cast results in a smoother surface that does not snag clothing or abrade the skin.

The term “splint” or “limb setting device” refers to a device used for support or immobilization of a limb, body part, or the spine. Accordingly, such device can be used in multiple situations, including temporary immobilization of potentially broken bones, soft tissue injury, damaged joints, and/or support for joints during physical activity

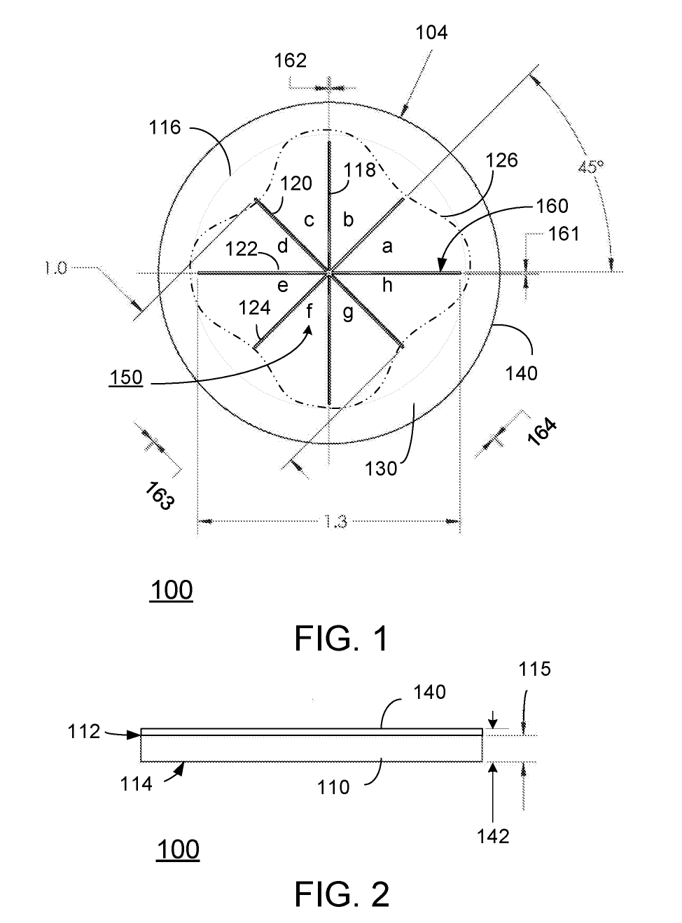

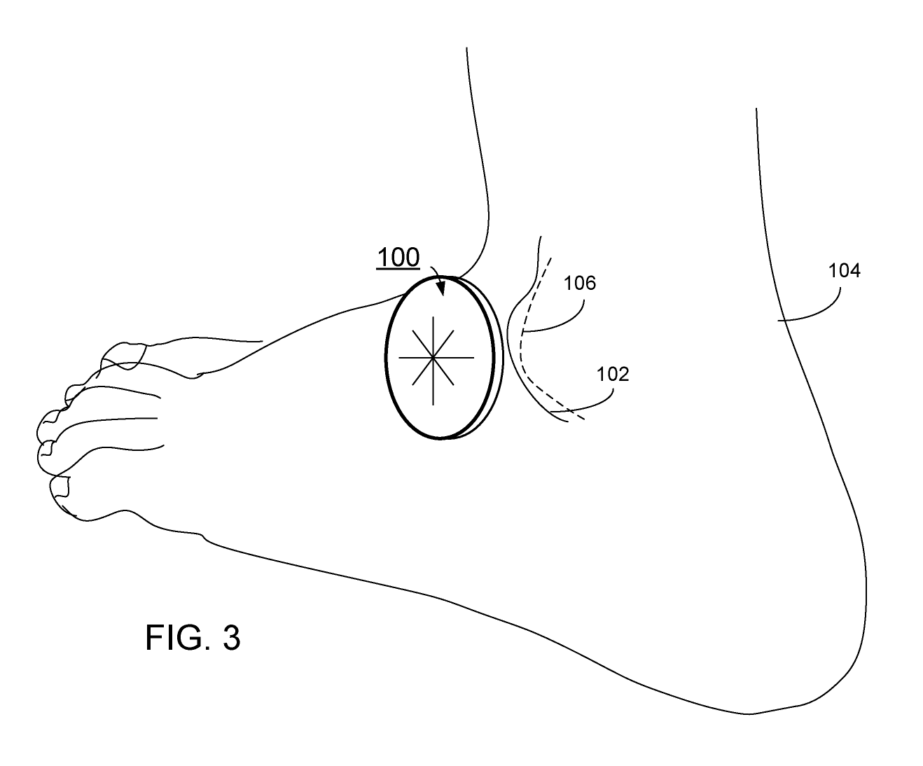

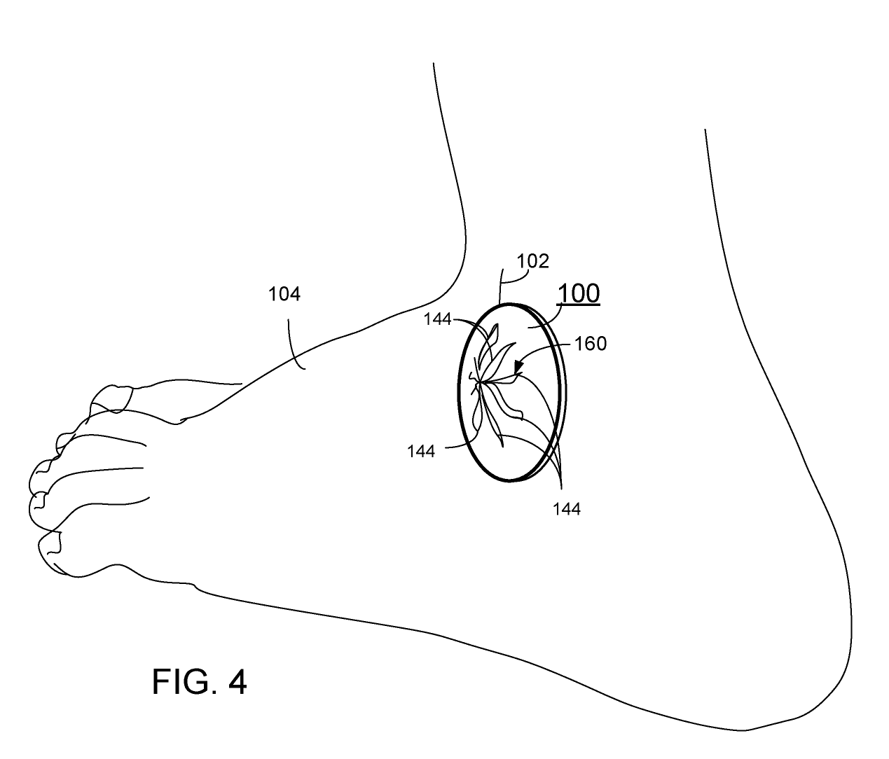

As is illustrated in FIGS. 1 through 4, an embodiment of the pressure relieving pad is generally shown by element 100 for use on a bony prominence 102 of the body. According to an embodiment of the present invention, the bony prominence 102 may be on the foot 104 such as, for example, an ankle bony prominence termed the talus 106 (also called the ankle bone) as shown in FIGS. 3 and 4. Although it should be appreciated by one who understands medical procedures and the operation of casting that the pressure relieving pad 100 of the present invention may be applied and used in castings and/or splinting with bony prominences of the body and limbs thereof

The pressure relieving pad 100 comprises a base substrate 110 as the pressure relieving pad 100 as a stand-alone an article of manufacture to affix to an edge, bony prominence or other body and used in castings and/or splinting with bony prominences of the body and limbs thereof According to the non-limiting embodiment of the present invention illustrating use in castings and/or splinting with bony prominences of the ankle bone of the body part and limbs thereof, the base substrate 110 is configured to be combined with a fibrous substrate 140 so as to provide the pressure relieving pad 100 article of manufacture to affix to a body part used in castings and/or splinting with bony prominences of the body and limbs thereof The construction of the pressure relieving pad 100 has the fibrous substrate 140 affixed, fastened, secured, united, attached physically, or otherwise connected as an associated part to an upper surface 112 of the base substrate 110, whereby these separate components are attached to, and have a functional relationship with, each other. The lower surface 114 of the base substrate 110 may be formed to be tacky so as to have a property of adhesiveness when applied to the skin. A predetermined pattern 150 may be formed in the pressure relieving pad 100 as is illustrated in FIGS. 1, 3 and 4. A person skilled in the medical field and the casting and/or splinting art will appreciate that the predetermined pattern 150 may be formed by single or multiple cuts such as, for example, a single cut, a cross-cut or a star-cut in the cut line should be non-limiting. While the present invention is described in a star-cut for the predetermined pattern 150, a single-cut design for the predetermined pattern 150 of the pressure relieving pad 100 has been manufactured easily that provides the advantages and results according to an embodiment of the present invention.

The base substrate 110 of gel material may be formed in numerous variations including different shapes, dimensions, be formed in varying thicknesses. The base substrate 110 can be formed from suitable materials such as, for example, a layer a medical grade thermo plastic composite gel material that has a spongy resilient characteristic. Suitable materials for the base substrate 110 of thermos plastic gel material with an adhesive property may be obtained in the market under the tradename Phase 4 Gel® manufactured by Innovative Medical Products, Inc. part number 715, may be formulated and constructed with an inherent “tackiness” to the gel material using suitable additives that may be varied and can be tailored to the specific clinical application of the pressure relieving pad 100. The base substrate 110 of Phase 4 Gel material may be formed in materials of various resilience. The base substrate 110 may have a thickness 115 that is suitable to relieve pressure under castings and/or splints such as, for example, approximately ranging from 2 to 4 mm.

The layer of fibrous material 140 can be formed from suitable materials such as, for example, fabric, bacteria resistant poly-fibers or other suitable material. The layer of fibrous material 140 may be loop substrate or fabric manufactured by Velcro, and available from Velcro B.V.B.A part number 158857. The layer of fibrous material 140 may be formed in a thickness 142 that is suitable to relieve pressure under castings and/or splints such as, for example, approximately ranging from 0.5 to 1 mm. One aspect of the fibrous material 140 is that it forms a structural backing to the base substrate 110. Another aspect is that the fibrous material 140 provides support to hold the base substrate 110 in a particular predetermined pattern 150.

According to an embodiment of the present invention, as shown in FIG. 1, the predetermined pattern 150 may be a unique design formed in the pressure relieving pad 100. An For example, the predetermined pattern 150 may be formed with a single or multiple cut incision with advantages including fit, forming to the bony prominence, improved casting, and air-flow (e.g. ventilation, perspiration relief). According to an embodiment of the present invention, the predetermined pattern 150 may be formed in a star-shaped incision through the central portion 116 at, in, or near the center of the base substrate 110 and fibrous material 140, as shown in FIGS. 1 and 3-4. Once cut, the pressure relieving pad 100 comprises one or more of cuts 118, 120, 122, and 124 attached around the base substrate 110, and free to move centrally. It is to be appreciated that the predetermined pattern 150 of the pressure relieving pad 100 may be formed to vary the number of the one or more cuts 118, 120, 122, and 124, depending on the application.

As shown in FIG. 1, the pressure relieving pad 100 may be configured with a single cut (e.g. cut-line 118), or multiple cuts such as, for example, a cross-cut (e.g. cut lines 118 and 122) or a star cut (e.g. cuts 18, 120, 122, and 124), whereby the cuts 118, 120, 122, and 124 from one or more leaves a, b, c, d, e, f, g, and h. The cuts 118, 120, 122, and 124 may be formed of the same length or of varying dimensions such as, for example, cuts 118 and 122 may be formed longer (e.g. 1.3 inches) than cuts 120 and 124 (e.g. 1.0 inches). Moreover, cuts 118, 120, 122, and 124 remove material such as, for example, approximately 0.005 mm to form a ventilated channel 160. In this manner, a structural support area 126 may be formed to add an area 126 of material inward of the outer circumference ring 130 so as to have structural integrity when applied to the bony prominence 102 as is shown in FIG. 4.

According to the present embodiment, the configuration of the one or more cuts 118, 120, 122, and 124 allows advantageously that the Phase 4 Gel may be configured to open up centrally retaining an outer support area ring or outer circumference ring 130 when placed over a bony prominence 102. In this manner, the present invention provides advantages as the pressure relieving the 100 may be arranged on the bony prominence 102 as shown in FIGS. 3 and 4 thereby providing greater thickness of gel at the periphery of the pad, rather than over the bony prominence 102 itself, which is a disadvantage of conventional fabrics and wrapping methods in cast and splints.

According to an embodiment of the present invention, as shown in FIGS. 1 and 4, the single or multiple cuts of the predetermined pattern 150 creates one or more ventilation opening(s) 160 providing for evaporation of perspiration beneath the pressure relieving pad 100. For example, the star-shaped incision of the predetermined pattern 150 has additional benefits including creating ventilation openings 161-164 between the cuts 118-124 of the pressure relieving pad 100 providing for evaporation of perspiration beneath the pressure relieving pad 100 as shown in FIG. 4. Perspiration is relieved within the cast through the ventilation opening(s) 160 formed as the leaves a, b, c, d, e, f, g, and h spread on the bony prominence 102 providing air-flow and air circulation. According to alternative embodiments of the present invention, perspiration also is relieved within the cast through the ventilation opening(s) 160 formed by a single cut 118 (e.g. two leaves forming a joined connection between sections a-b and d-g without interleaving spaces, or a cross-cut 118 and 122 (e.g. four leaves formed from joined sections a-b, c-d, e-f, and g-h put or brought together so as to form a unit).

Consequently, according to the various embodiments of the present invention, less moisture builds-up in a cast and/or splint under the pressure relieving pad 100 thereby inhibiting bacterial growth and providing greater comfort to the patient. Consequently, the pressure relieving pad 100 of the present invention has one or more improvements over conventional constructions including, but not limited to, as follows:

1. The gel material does not absorb perspiration.

2. The pressure relieving pad 100 does not harbor odor causing bacteria.

3. The pressure relieving pad 100 maintains its shape and does not compact down over time reducing its ability to provide padding to a wide range of bony prominence dimensions.

4. The single or multiple cuts of the predetermined pattern 150 allows the pressure relieving pad 100 to conform to a wide range of bony prominence dimensions.

5. The single and multiple cut of the predetermined pattern 150 opens up over the prominence and this reduces the amount of material directly over the prominence, shifting the pressure to the peripheral edges of the pad, and relieving pressure on the bony prominence.

6. The ventilation channel 160, shown as opening(s) 161, 162, 163, and 164 of FIG. 1, from the cut in the predetermined pattern 150 (e.g. a single cut or multiple cuts such as the star cut design) allows for air flow, between the body, the cast and the pressure relieving pad 100, to reduce the build-up of perspiration under the pressure relieving pad 100.

7. The pressure relieving pad 100 fibrous substrate 140 of the pressure relieving pad 100 backing is tough and resistant to being cut by a cast saw, protecting the underlying bony prominence.

8. The base substrate 110 made from gel material with adhesive properties, for example, Phase 4 Gel, absorbs heat and liquefies from the heat of the cast saw, preventing the heat of the blade of the saw from burning the skin of the patient.

9. The sticky, adhesive quality of the gel material allows the pad to adhere to the skin and the fibrous substrate 140 meshes with the cast padding to keep it in place within the cast, for example, beneath the fiberglass cast material.

10. The pressure relieving pad 100 reduces the amount of standard cast padding, which would compress over time, resulting in a cast that will remain snug.

11. The pressure relieving pad 100 may have application for “chafing protection” in “splint” or “limb setting device”.

12. The ability to make the pressure relieving pad 100 in specific shapes for various applications under a cast, such as around the base of the thumb or at the edge of a cast to protect the skin.

13 A thinner gel the pressure relieving pad 100 may have other applications and usefulness, for example, as an edge padding.

14. Additives, for example, of “deodorant” perfume or other substances may be combined to the base substrate 110 of the gel of the pressure relieving pad 100 may have application and usefulness to reduce cast smell.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An article of manufacture for casting a body part, the article comprising:
a base substrate of a predetermined design, said base substrate comprising a gel material with a thickness suitable to relieve pressure under castings and/or splints and with an adhesive quality adapted to affix to a bony prominence of the body part; and
at least one ventilation channel cut through the base substrate of gel material functioning to increase the padding around the base of the prominence thereby providing extended comfort and fitting of a rigid splint, cast or other fixing device.

2. The article of claim 1 further comprising a fibrous substrate operably connected to said base substrate, wherein said at least one ventilation channel cut through the gel and fibrous substrate functioning to increase the padding around the base of the prominence thereby providing extended comfort and fitting of a rigid splint, cast or other fixing device

3. The article of claim 1 further comprising wherein said fibrous substrate and base pad formed with a predetermined design.

4. The article of claim 3 wherein said predetermined design is a single ventilation channel pressure relieving pad.

5. The article of claim 3 wherein said predetermined design is a cross-cut ventilation channel pressure relieving pad.

6. The article of claim 32 wherein said predetermined design is a cross-cut ventilation channel pressure relieving pad.

7. The article of claim 1 wherein said gel is selected from material from the group of thermo plastic gel material.
